(19) United States
(12) Patent Application Publication
(10) Pub. No.: US 2007/0100461 A1
(43) Pub. Date: May 3, 2007

(54) Knee prosthesis

(75) Inventors: Stephen Incavo, Houston, TX (US); Bruce D. Beynnon, South Burlington, VT (US)

Correspondence Address: EDWARDS & ANGELL, LLP, P.O. BOX 55874, BOSTON, MA, 02205, US

(73) Assignee: The University of Vermont and State Agriculture College, Burlington, VT (US)

(21) Appl. No.: 11/403,526

(22) Filed: Apr. 12, 2006

Related U.S. Application Data
(60) Provisional application No. 60/670,813, filed on Apr. 12, 2005.

(51) Int. Cl. A61F 2/38

(52) U.S. Cl. 623/20.19; 623/20.34

(57) ABSTRACT

The invention relates to an orthopedic prosthetic device and a method of implanting such device. More particularly, the invention relates to a knee replacement prosthesis having femoral, patellar, and tibial components having surfaces for receiving bone cement and surfaces which promotes bone ingrowth.

### RELATED APPLICATIONS

This application claims priority to U.S. Provisional Application Ser. No. 60/670,813, filed Apr. 12, 2005.

### BACKGROUND OF THE INVENTION

Arthritis of the knee joint is not only painful but can be permanently debilitating. With ever increasing frequency, doctors are replacing arthritic knees with prosthetic devices having a tibia component, a femur component and a patella component which mimic the articulation between the tibia and the femur. A complete knee replacement is often referred to as a Total Knee Arthroplasty (TKA). It is a primary goal of TKA to provide a stable, pain free and long lasting knee replacement.

Fixation of the tibia, femur and patella components of a prothesis during implantation has customarily involved either bone cement or natural bone ingrowth. Orthopedic surgeons typically prefer cementless fixation for what is considered to be its potential to provide long term implant stability. Knee replacement prothesis wherein fixation is accomplished through bone ingrowth may have a porous layer to facilitate bone ingrowth. For instance, U.S. Pat. No. 4,479,271 discloses a tibial component which utilizes a fibrous metal mesh layer to facilitate bone ingrowth. U.S. Pat. Nos. 3,605,123; 3,855,638; and 4,550,448 further disclose porous layers which aid in the development of bone ingrowth.

Three factors are thought of as important for achieving optimal bone ingrowth: (1) close contact between bone and the prothesis, (2) the absence of micromotion and (3) the elimination of any effect that would inhibit bone growth. (see, Voltz, R. G.; Nisbet, J. K.; Lee, R. W.; and McMurtry, M. G.: The Mechanical Stability of Various Noncemented Tibial Components., Clin. Orthop. and Rel. Res., 226:38-42, 1988). Loosening due to micromotion of the tibial component is the most frequent cause of long term TKA failure. Porous coated implant designs were thought to have solved the loosening problem by providing a stable implant fixation through bony ingrowth. (see, Shimakagi, H.; Bechtold, J. E.; Sherman, R. E.; and Gustilo, R. B.: Stability of the Tibial Component in Cementless Total Knee Arthroplasty., J. of Orthopaedic Res., 8:64-71, 1990). However, cancellous bone ingrowth in the tibial prosthesis has been unpredictable. (see, Branson, P. J.; Steege, J. W.; Wixson, R. L.; Lewis, J.; and Stulberg, S. D.: Rigidity of Initial Fixation with Uncemented Tibial Knee Implants., J. of Arthroplasty, 4:21-26, 1989).

Cemented TKA prosthesis designs have met with more clinical success and a lower incidence of loosening due to micromotion. (see, Krackow, K. A.; Hungeford, D. S.; Trnka, H. J.; Maar, D. C.; Mont, M. A.; and Urquhart, M.: Cemented Versus Uncemented Primary Total Knee Arthroplasty: A Comparative Study of the First 100 Subjects in Each Group., Read at the Annual Meeting of the American Academy of Orthopaedic Surgeons, Washington D.C., Feb. 20, 1992; Insall, J. N.; Binazzi, R.; Soudry, M.; and Mestriner, L. A.: Total Knee Arthroplasty., Clin. Orthrop. and Rel. Res., 192:13-22, 1985; and Walker, P. S.: Requirements for Successful Total Knee Replacements, Orthrop., Clin. of North Am., 20:15-29, 1989). Yet, even bone cement fixated prosthetic components are susceptible to loosening.

Hybrid knee replacement prosthetic components have been developed in an attempt to overcome some of the above described disadvantages of the cementless and bone cement prosthesis. Hybrid components utilize both cement and bone ingrowth for fixation. An example of such a hybrid prosthesis component is disclosed in U.S. Pat. No. 4,938,769 to Shaw. The Shaw prosthesis suffers from disadvantages primarily resulting from the placement of the porous bone ingrowth material on the central stem and pegs. The central stem and pegs of the Shaw prosthesis are structured to fit within the tibia itself and promote bone ingrowth between the bone tissue of the interior of the tibia and the central stem and pegs. While this design would appear to effectively promote bone ingrowth, because bone ingrowth occurs about the central stem and pegs, removal of the tibial prosthesis (as a result of infection) would cause excessive tibia damage. Moreover, the bone ingrowth area is of limited surface area and does not effectively utilize the larger surface area of the upper end of the tibia for bone ingrowth.

### BRIEF SUMMARY

The invention provides, in one aspect, a knee replacement prosthesis comprising a femoral component of generally concave shape. The femoral component has a bone facing surface and an anatomically shaped articular surface having anterior and posterior ends, wherein the posterior end forms two condyles along an axis of the femoral component, wherein the bone facing surface comprises at least one stem, a surface for receiving a fixation material adjacent to the anterior and posterior ends, and a central surface for promoting bone ingrowth.

In one aspect, the invention provides a knee replacement prosthesis comprising a femoral component of generally concave shape. The femoral component comprises a bone facing surface and an anatomically shaped articular surface having anterior and posterior ends, wherein the posterior end forms two condyles along an axis of the femoral component, wherein the bone facing surface comprises at least one stem, a surface for receiving a fixation material along a perimeter of the bone facing surface and a central surface for promoting bone ingrowth.

In one embodiment, the prosthesis further comprises a tibial component, which comprises a laterally extending plate having a proximal surface and a distal surface, the distal surface including a peripheral surface for receiving fixation material and a planar central surface which promotes bone ingrowth, the peripheral surface and the planar central surface each having surface areas which are substantially equal; and a central stem extending from the distal surface and being substantially perpendicular thereto.

In another embodiment, the prosthesis further comprises a patellar component. The patellar component, may comprise a substantially circular plate having a patellar facing surface, wherein the patellar facing surface has a peripheral surface for receiving a fixation material and a central surface for promoting bone ingrowth. In a related embodiment, the patellar component may comprise a substantially circular plate having a patellar facing surface, wherein the patellar facing surface has a peripheral surface for promoting bone ingrowth and a central surface for receiving a fixation material. In yet another related embodiment, the patellar component may comprise a substantially circular plate having a patellar facing surface, wherein the patellar facing has a one or more pegs and a surface for promoting bone ingrowth.

In certain embodiments, the planar central surface and the central surface have bone ingrowth promoting material.

In one embodiment, the femoral component further comprises a partition continuously extending around a perimeter of the bone facing surface which separates the surface for receiving a fixation material and the central surface.

In one aspect, the invention provides, a knee replacement prosthesis comprising a patellar component comprising a substantially circular plate having a patellar facing surface, wherein the patellar facing surface has a peripheral surface for receiving a fixation material and a central surface for promoting bone ingrowth.

In one aspect, the invention provides a knee replacement prosthesis comprising a patellar component comprising a substantially circular plate having a patellar facing surface, wherein the patellar facing surface has a peripheral surface for promoting bone ingrowth and a central surface for receiving a fixation material.

In certain embodiments, the patellar component further comprises a partition continuously extending around the circular plate separating the peripheral surface from the planar central surface.

In one aspect, the invention provides, a knee replacement prosthesis comprising a patellar component comprising a substantially circular plate having a patellar facing surface, wherein the patellar facing has a one or more pegs and a surface for promoting bone ingrowth.

The invention also provides, in one aspect, a method for implanting knee replacement prosthesis, comprising surgically exposing the knee joint of a subject, preparing the surface of the femur for implantation, and securing a femoral component of generally concave shape to the femur of the subject, wherein the femoral component comprises a bone facing surface and an anatomically shaped articular surface having anterior and posterior ends, wherein the posterior end forms two condyles along an axis of the femoral component, wherein the bone facing surface comprises at least one stem, a surface for receiving a fixation material adjacent to the anterior and posterior ends, and a central surface for promoting bone ingrowth.

The invention also provides, in one aspect a method for implanting a knee replacement prosthesis comprising preparing a surface of the patella for implantation, securing a patellar component of the invention to the patella.

The methods of the invention, may also further comprise preparing the surface of the tibia for implantation; and securing a tibial component to the tibia, wherein the tibial component comprises a laterally extending plate having a proximal surface and a distal surface, the distal surface including a peripheral surface for receiving fixation material and a planar central surface which promotes bone ingrowth, the peripheral surface and the planar central surface each having surface areas which are substantially equal; and a central stem extending from the distal surface and being substantially perpendicular thereto.

A method aspect of the invention, comprises surgically exposing the knee joint of a subject, preparing the surface of the femur for implantation, and securing a femoral component of generally concave shape to the femur of the subject, wherein the femoral component comprises a bone facing surface and an anatomically shaped articular surface having anterior and posterior ends, wherein the posterior end forms two condyles along an axis of the femoral component, wherein the bone facing surface comprises at least one stem, a surface for receiving a fixation material along a perimeter of the bone facing surface and a central surface for promoting bone ingrowth.

In one aspect, provided herein are methods for implanting knee replacement prosthesis, comprising surgically exposing the knee joint of a subject, preparing the surface of the femur for implantation, and securing a femoral component of generally concave shape to the femur of the subject, wherein the femoral component comprises a bone facing surface and an anatomically shaped articular surface having anterior and posterior ends, wherein the posterior end forms two condyles along an axis of the femoral component, wherein the bone facing surface comprises at least one stem, a surface for receiving a fixation material adjacent to the anterior and posterior ends, and a central surface for promoting bone ingrowth.

In one aspect, the methods may further comprise preparing a surface of the patella for implantation, securing a patellar component to the patella, wherein the patellar component comprises a component of any one of the implants described herein.

In one aspect, the methods may further comprise preparing the surface of the tibia for implantation; and securing a tibial component to the tibia, wherein the tibial component comprises a laterally extending plate having a proximal surface and a distal surface, the distal surface including a peripheral surface for receiving fixation material and a planar central surface which promotes bone ingrowth, the peripheral surface and the planar central surface each having surface areas which are substantially equal; and a central stem extending from the distal surface and being substantially perpendicular thereto.

In one embodiment, the preparing the surface of the tibia comprises cutting an upper end of a tibia of a subject in a shape to accommodate a shape of the tibial component; and fitting a tibial baseplate template into the upper end of the tibia to form a slot to receive the tibial component;

In one embodiment, the securing a tibial component to the tibia comprises:

applying a fixation material to the peripheral surface for receiving fixation material; and pressing the tibial component into the upper end of the tibia.

In one embodiment, the preparing the surface of the femur for implantation comprises cutting a distal end of the femur to the dimensions of the femoral component.

In one embodiment, the securing a femoral component comprises applying a fixation material to the surface for receiving fixation material; and pressing the femoral component onto the femur.

In one aspect, provided herein are methods for implanting knee replacement prosthesis, comprising surgically exposing the knee joint of a subject, preparing the surface of the femur for implantation, and securing a femoral component of generally concave shape to the femur of the subject, wherein the femoral component comprises a bone facing surface and an anatomically shaped articular surface having anterior and posterior ends, wherein the posterior end forms two condyles along an axis of the femoral component, wherein the bone facing surface comprises at least one stem, a surface for receiving a fixation material along a perimeter of the bone facing surface and a central surface for promoting bone ingrowth.

In one embodiment, the methods may further comprise preparing a surface of the patella for implantation, securing a patellar component to the patella, wherein the patellar component comprises a component of any one of the implants described herein.

In one embodiment, the methods may further comprise preparing the surface of the tibia for implantation, and securing a tibial component to the tibia, wherein the tibial component comprises a laterally extending plate having a proximal surface and a distal surface, the distal surface including a peripheral surface for receiving fixation material and a planar central surface which promotes bone ingrowth, the peripheral surface and the planar central surface each having surface areas which are substantially equal; and a central stem extending from the distal surface and being substantially perpendicular thereto.

In one embodiment, the preparing the surface of the tibia comprise cutting an upper end of a tibia of a subject in a shape to accommodate a shape of the tibial component; and fitting a tibial baseplate template into the upper end of the tibia to form a slot to receive the tibial component;

In one embodiment, the securing a tibial component to the tibia comprises applying a fixation material to the peripheral surface for receiving fixation material; and pressing the tibial component into the upper end of the tibia.

In one embodiment, the preparing the surface of the femur for implantation comprises cutting a distal end of the femur to the dimensions of the femoral component.

In one embodiment, the securing a femoral component comprises applying a fixation material to the surface for receiving fixation material; and pressing the femoral component onto the femur.

Other embodiments of the invention are disclosed infra.

### BRIEF DESCRIPTION OF THE DRAWINGS

FIGS. 1A and 1B are a perspective views of a femoral component.

FIG. 2 is a perspective view of a femoral component.

FIG. 3 is a perspective view of the condyles of the femoral component.

FIGS. 4A and 4B are side and front views, respectively, of a femoral component.

FIG. 5 is a bottom view of a patellar component.

FIG. 5 is a bottom view of a patellar component.

FIG. 6 is a bottom view of a patellar component.

FIG. 7 is a bottom view of a patellar component with stems.

FIG. 8 is a side view of a patellar component.

FIG. 9 is a top view of a first embodiment of the tibial tray.

FIG. 10 is a bottom view of the first embodiment of the tibial tray.

FIG. 11 is a side view of the first embodiment of the tibial tray.

FIG. 12 is a front view of the first embodiment of the tibial tray.

FIG. 13 is a top view of a second embodiment of the tibial tray.

FIG. 14 is a bottom view of the second embodiment of the tibial tray.

FIG. 15 is a side view of the second embodiment of the tibial tray taken along lines 7-7 of FIG. 14.

FIG. 16 is a side view of the second embodiment of the tibial tray taken along lines 8-8 of FIG. 14.

### DETAILED DESCRIPTION

Disclosed herein are novel, long-lasting, and well-fitting prosthetic components for the knee. Further disclosed herein are method for implanting the prosthetic components. The components of the invention overcome the disadvantages of previously known devices, for example, the unique combinations of bone ingrowth surfaces and the use of fixation materials provide advantages for installation and for long-term stability of the components. These advantages lead to overall clinical success for the patient.

With reference to the figures where like elements have been given like numerical designation to facilitate an understanding of the present invention.

Femoral Component

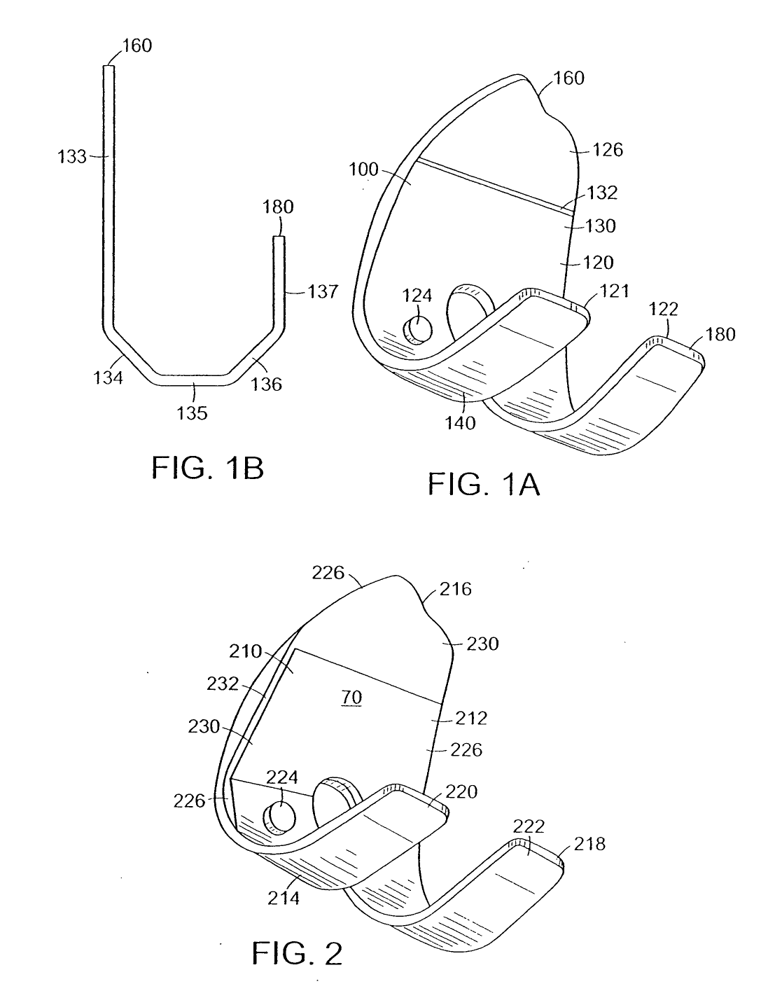

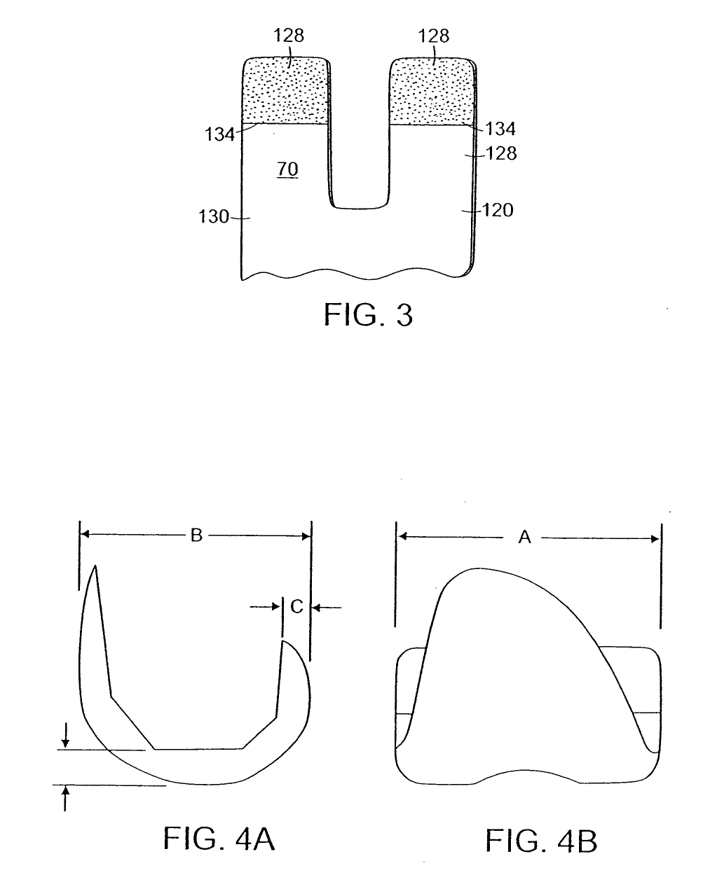

FIGS. 1A and 1B are a side and perspective views of a femoral component 100 of generally concave shape, respectively. The femoral component has a bone facing surface 120 and an anatomically shaped articular surface 140 having anterior 160 and posterior 180 ends. The posterior end forms two condyles 121, 122 along an axis of the femoral component. The bone facing surface has a stem 124. The bone facing surface also has a surface for receiving a fixation material 126, 128 (FIG. 3) adjacent to the anterior and posterior ends and a central surface 130 for promoting bone ingrowth. Partitions 132, 134 (FIG. 3) separate the surface for receiving fixation material 126, 128 (FIG. 3) from the central surface 130. In this example, there are five surfaces of the femoral component that oppose the bone. These include, 1) the anterior portion 133, 2) the anterior chamfer 134, 3) distal portion 135, 4) posterior chamfer 136, and 5) the posterior portion 137. Any combination of these surfaces may used for fixation of the component. For example, two chamfered surfaces could receive a fixation material with the other three surfaces not receiving fixation material. In another example, the distal portions receive fixation material while the other three surfaces do not receive the material. The surfaces that do not receive the material may or may not, in any combination, have bone in-growth surfaces. Some references as noted above are to FIG. 3 above, which is a perspective view of the condyles of a femoral component.

FIG. 2 is a perspective view of a femoral component 210 of generally concave shape. The femoral component has a bone facing surface 212 and an anatomically shaped articular surface 214 having anterior 216 and posterior ends 218. The posterior end forms two condyles 220, 222 along an axis of the femoral component.

The condyles may extend from between about 10 to about 50 mm from the posterior end of the femoral component. The bone facing surface 212 has two stems 224, a surface for receiving a fixation material 226 along a perimeter of the bone facing surface and a central surface 230 for promoting bone ingrowth. The central surface 70 has bone ingrowth promoting material. Peripheral surface 226 receives a fixation material during implantation of the femoral component 210 and affords close apposition between the cancellous bone and component. Fixation material at and around the peripheral surface 226 of the component permits rigid fixation equal to that of traditional cement prothesis. The use of bone cement at peripheral surface aids the ingrowth process by providing a more even surface for the normal loading of the femur to be distributed.

The shape of the femoral component is intended to fit the lower end of a subject's femur. The bone facing surface is intended to be placed against the end of the subjects femur and fixed thereto during installation. The stems are intended to fit into the subject's femur. The stem may, for example, have a uniform diameter or be tapered. It may have a cylindrical side wall and circular distal end. Other configurations are also possible and will be readily known to one of skill in the art having the benefit of this disclosure. The stem or stems are adapted to reduce the micro-motion and contribute to bone ingrowth. The stem has an optional bone ingrowth surface or is adapted to receive a fixation material during installation.

The partition along the perimeter is continuously extending around a perimeter of the bone facing surface. The partition separates the surface for receiving a fixation material and the central surface. The partition is operative to prevent fixation material from flowing into the central surface. Partition configurations are described in detail infra.

Generally, the dimensions of the formal components will be illustrated with respect to FIG. 4. The A dimension will generally be from between about 55 to about 90 cm. The B dimension will generally be from between about 45 to about 85 cm and the C dimensions will generally be from between about 6 to about 11 cm. The C dimensions may be the same or they may vary from one another. The dimensions of the femoral component may vary depending on the age of the subject, the size of the subject, the activity of the subject before surgery and anticipated activity level post-surgery, and the subject's bone density.

Patellar Component

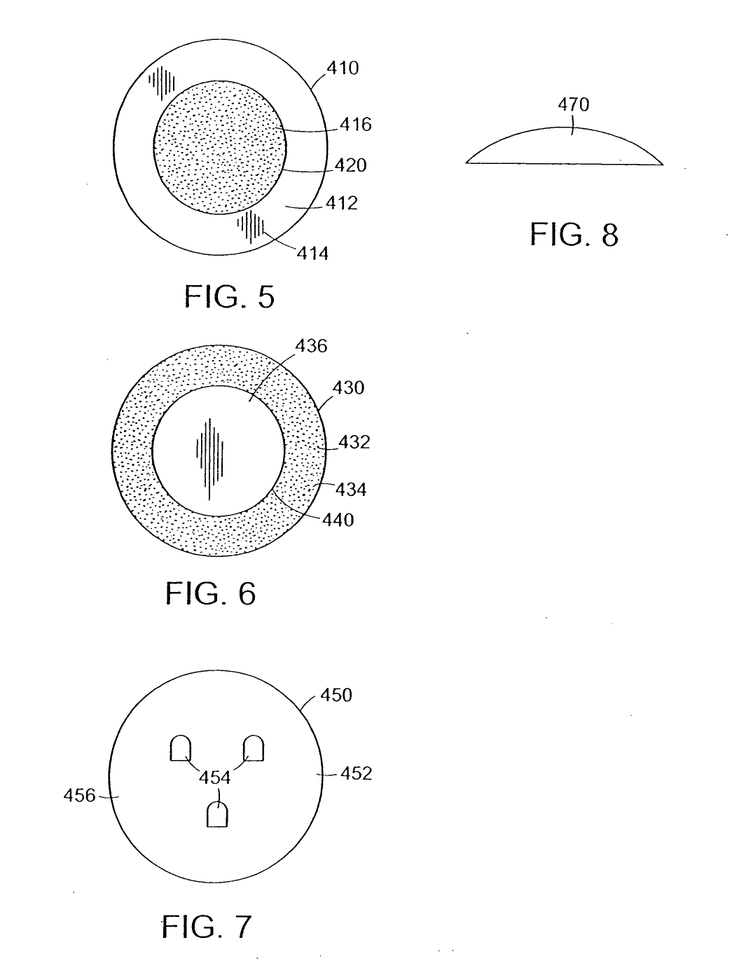

FIG. 5 is a top view of a patellar component 410. The patellar component 410 is a substantially circular plate having a patellar facing surface 412. The patellar facing surface 412 has a peripheral surface 414 for receiving a fixation material and a central surface 416 for promoting bone ingrowth. The patellar component has a partition 420 continuously extending around the circular plate separating the peripheral surface from the planar central surface.

FIG. 6 is a top view of a patellar component 430. The patellar component 430 is a substantially circular plate having a patellar facing surface 432, wherein the patellar facing surface 432 has a peripheral surface 434 for promoting bone ingrowth and a central surface 436 for receiving a fixation material. The patellar component has a partition 440 continuously extending around the circular plate separating the peripheral surface from the planar central surface.

In certain embodiments of the patellar component, the surface for receiving a fixation material and the central surface are substantially equal in area. Alternately, the surface for receiving a fixation material is about ⅓ to about ½ of the bone facing surface. The optimal amount of each surface for the patellar component may be determined in part, by the size of the patella, the extent of damage to the patella or other portion of the knee being repaired, the activity level of the subject, etc. In certain embodiments, the surface for receiving a fixation material is a recessed pocket.

FIG. 7 is a top view of a patellar component 450. The patellar component 450 is a substantially circular plate having a patellar facing surface 452. The patellar facing 452 has three pegs 454 and a surface for promoting bone ingrowth 456. There may be one, two, three, four, or five pegs on patellar components. The pegs may have a any shape (e.g., cone, circular, or oval). The pegs may be from between about 1-8 mm in height. The pegs may also be from between about 1 and 10 mm in width. The height and width may depend in part on the size of the patella, the age of the subject, and/or the damage to the patella. The pegs may be attached to the bone with a fixation material. The pegs may also have a bone ingrowth surface. FIG. 8 is a side view of FIGS. 5 and 6. This shows an exemplary curvature of the surface not facing the bone.

Patellar components may be circular plates having heights in a range of 3.0 to 13 mm. Circular plate has a circumference of between about 20 to about 45 mm.

The patellar components of the invention may also have other shapes, for example, they may take on the shape of a patella (e.g., oblong) or they may be square. The outer facing surface of the patellar component may be spherical, may have one or more grooves, or may be shaped like the subject's own patella.

Tibial Component

In reference to the tibial tray of the present invention illustrated in FIGS. -9-12, the hybrid tibial tray 10 may include a laterally extending plate 11. Plate 11 is preferably shaped to fit the upper end of a subject's tibia. It is especially preferred if plate 11 has a height in the range of 3.0 to 7.0 mm.

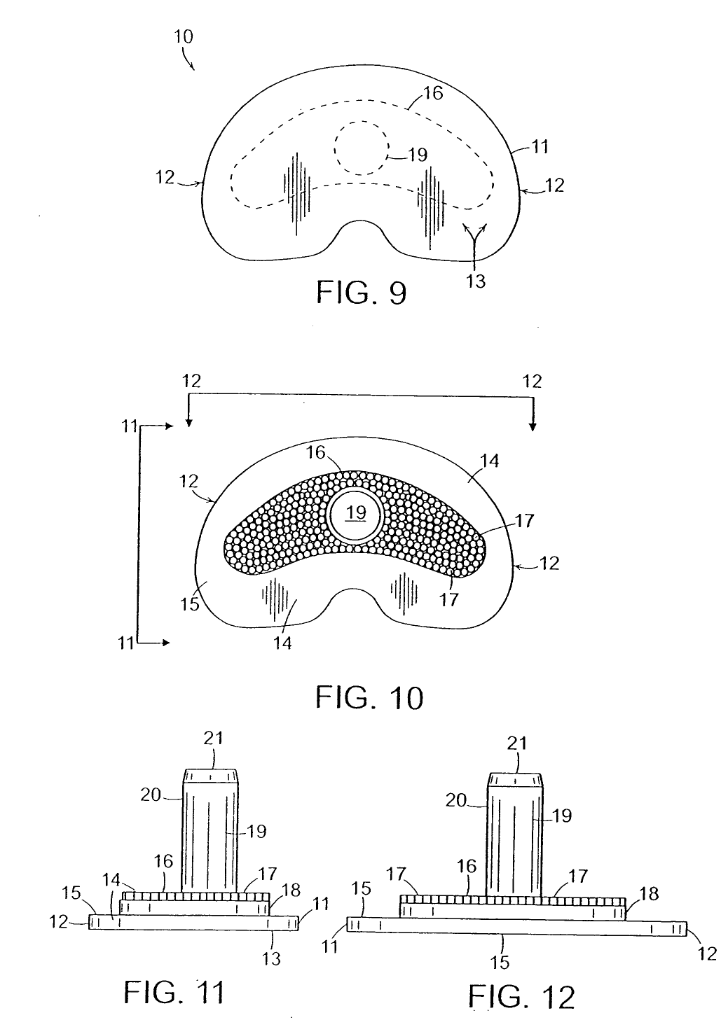

As shown in FIGS. 9 and 11, plate 11 may have a partition in the form of a peripheral shoulder 12. Preferably, plate 11 has a width of 80 mm and a length of 53 mm. Plate 11 may also have a proximal surface 13 that receives the femur component of the knee replacement prosthesis. It is preferred that proximal surface 13 be generally flat. The beaded surface shown in the figures, including FIG. 9 may alternately be a roughen metal surface or a ceramic surface, for example, a bone on growth surface.

With reference to FIGS. 10 and 14, plate 11 may have a distal surface 14, which is intended to be placed against the upper end of a subject's tibia and fixed thereto during implantation of hybrid tibial tray 10. Distal surface 14 preferably includes a peripheral surface 15 for receiving bone cement and a central surface 16, which promotes bone ingrowth.

It is desirable if peripheral surface 15 has a diameter of 7 mm at the front side of hybrid tibial tray 10, 12 mm at the rear side of hybrid tibial tray 10, and 8 mm at each of the sides of hybrid tibial tray 10. Peripheral surface 15 receives bone cement upon implantation of hybrid tibial tray 10 and affords close apposition between the tibial cancellous bone and central surface 16. Bone cement at and around peripheral surface 15 of hybrid tibial tray 10 permits rigid fixation equal to that of traditional cement prothesis. The use of bone cement at peripheral surface 15 aids the ingrowth process by providing a more even surface for the normal axial loading of the tibia to be distributed. Even distribution of force is material to the reduction of micromotion and the success of bone ingrowth in central surface 16 of hybrid tibial tray 10.

Central surface 16 preferably is made of material that promotes bone ingrowth. For example, central surface 16 may be a porous material or have a surface or have a layer of porous material that promotes bone ingrowth. An example of such material is a fibrous metal mesh such as that taught in U.S. Pat. No. 4,479,271, the disclosure of which is incorporated herein by reference.

The central surface 16 may be coated with a bone ingrowth promoting material. The coating may form a porous layer 17.

As illustrated in FIGS. 11-14, hybrid tibial tray 10 may have a central stem 19 extending from distal surface 14 of plate 11. Central stem 19 is designed to fit within intramedullary canal of a subject's tibia when implanted. Preferably, central stem 19 has a cylindrically shaped side wall 20 and a circular distal end 21. Side wall 20 of central stem 19 may be uniform in diameter or tapered toward distal end 21. Central stem 19 further aids in reducing micro-motion and thus contributes to successful bone ingrowth. Plate 11 and central stem 19 may be constructed of a bio-compatible material such as medical grade titanium.

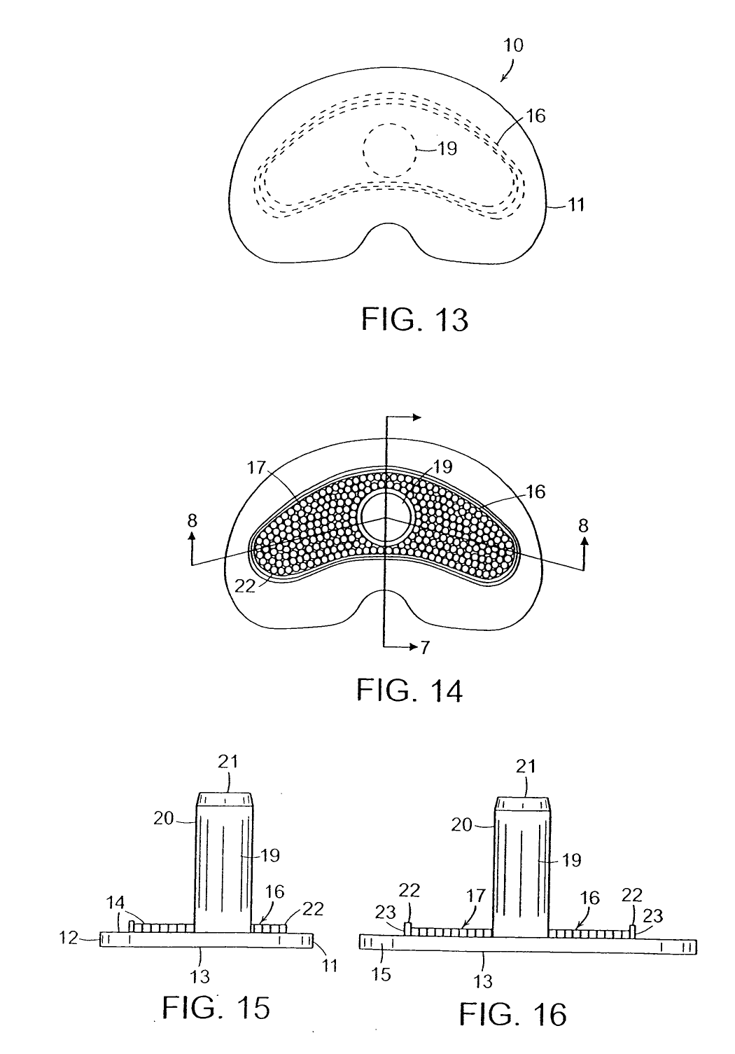

With reference to FIGS. 15 and 16, plate 11 may have a partition separating peripheral surface 15 from central surface 16. Preferably, the partition is a continuous structure, for example, a raised shoulder 22 which may completely surround central surface 16. It is especially desirably if raised shoulder 22 is configured as shown in FIGS. 12 and 13, with a V-shaped cross section 23 and having a height which is at least the height of porous layer 17. More preferably, raised shoulder 22 is of a height which is greater than the height of porous layer 17. It is also preferable for the height of raised shoulder 22 to be twice the height of plate 11. For example, raised shoulder 28 may have a height in the range of 0.6 to 2.1 mm or in the range of 6.0 to 14.0 mm.

Referring now to FIGS. 11 and 12, the partition may be in the form of stepped portion 18 containing central surface 16. In this configuration, central surface 16 is raised in relation to peripheral surface 15. It is preferred if stepped portion 18 has a height that is twice the height of plate 11. For example, stepped portion 18 may have a height in the range of 3.0 to 14.0 mm or more preferably in the range of 3.0 to 8.0 mm. A height of 6.0 mm is most preferred.

The surface area of the peripheral surface 15 and the surface area of central surface 16 are, in one embodiment, substantially equal.

Bone ingrowth promoting materials of the invention may be a porous layer. Examples of such porous layers according to the invention are found in U.S. Pat. Nos. 3,605,123; 3,855,638; 4,550,448; and 5,201,766, the disclosures of which are incorporated herein by reference. It is especially preferred if the porous layer 17 is composed of a plurality of metallic beads. Such beads are well known and are commercially available. Porous layer 17 desirably has a thickness in the range of 0.5 to 2.0 mm.

Other methods of producing such bone ingrowth promoting surfaces include providing a mass of titanium spheres vacuum fused onto the datum surface of the implant. This method is described in U.S. Pat. No. 4,834,756. A similar procedure is described in U.S. Pat. No. 4,644,942, wherein an extractable component and titanium spheres are densified as a coating, which is fused onto a datum surface of the implant, and the extractable component subsequently is extracted. Still other methods of providing bone ingrowth surfaces include the formation of perforated thin metallic sheets or plates by means of chemical milling and/or photo-chemical etching techniques as described in U.S. Pat. No. 3,359,192; U.S. Pat. No. 5,606,589; and U.S. Pat. No. 5,814,235.

The material forming the porous layer may have pores, for example, within a size range of about 10 microns to about 400 microns or greater. For example, the pore diameter can be in the range of about 1 to about 2 millimeters. Furthermore, the porosity of the porous portion may be in the range of from about 20% to about 50%. Moreover, the porous layer may have an open porous structure or a closed porous structure.

Porous layers may be composed of a plurality of beads, a roughened bone on growth surface, or of a ceramic coating. Porous layers may be from between about 0.5 to about 7 mm in thickness. Partitions, as used herein, are continuous or discontinuous structures, for example, a raised shoulder may completely surround a central surface. A partition may also divide off a section, such as a distal end. It is especially desirably if raised shoulder is configured with a V-shaped cross section and having a height which is at least the height of a porous layer. More preferably, a raised shoulder is of a height which is greater than the height of porous layer. It is also preferable for the height of raised shoulder to be twice the height of a plate or twice the height of the thickness of a component. For example, raised shoulder may have a height in the range of 0.6 to 2.1 mm or in the range of 6.0 to 14.0 mm.

Partitions may be in the form of stepped portion. For example, in this configuration the bone ingrowth portion is raised in relation to portion for receiving a fixation material. Stepped portions may have, for example, heights that are twice the height of plate or thickness of a component. For example, stepped portions may have a height in the range of 3.0 to 14.0 mm, 3.0 to 8.0 mm or 6-14 mm. Partitions of the invention, may have V-shaped cross sections. Exemplary components of the invention have a stepped portion with a height of 6.0 mm.

Fixation materials useful in the invention include cements such as PMMA. The fixation materials may be introduced onto the components of the prosthesis by hand or by use of an injector or other applicator. The fixation materials may contain additives, for example, antibiotics and antifungal agents.

In certain embodiments, the prostheses components may be manufactured from implantable grades of ultra-high molecular weight polyethylene and cobalt chromium molybdenum alloy. The components of the system would be manufactured from surgical grade stainless steels. It is within the concept of the present invention that the components may be manufactured from any implantable materials or surgically acceptable materials known in the art, such as, for example titanium alloys, ceramics, composites, or the like. In the manufacture of the instruments and devices of the system described herein, the components can be integrally formed or separately formed and assembled using permanent or temporary connections as are well known in the art. The prosthesis may be made of, for example, ceramic, aluminum oxide, zirconium oxide, metal, metal alloy, Co—Cr—W—Ni, Co—Cr-M, CoCr alloy, CoCr Molybdenum alloy, Cr—Ni—Mn alloy, powder metal alloy, 316L stainless steel, Ti 6AI-4V ELI, polymer, polyurethane, polyethylene, wear resistant polyethylene, cross-linked polyethylene, thermoplastic elastomer, biomaterial, polycaprolactone, diffusion hardened material, Ti-13-13, Zirconium, Niobium, porous coating system, hydrophilic coating, hydroxyapatite coating, and tri-calcium phosphate.

As used herein, subjects include mammal, for example, a human, horse, or a primate.

Implanting

Implanting or securing prosthetic knee components is well known in the art. Techniques such as those disclosed in U.S. Pat. No. 4,653,488, may be used.

Implanting the femoral components of the invention onto the femur of a subject include preparing the surface of the femur for implantation. This may be done by resurfacing or resecting the femur to form a resected articular surface having an anterior surface and a posterior surface each extending between a lateral side and a medial side (e.g., cutting a distal end of the femur to the dimensions of the femoral component). At least one of the anterior surface and posterior surface may be sloped relative to the other such that the anterior surface and posterior surface converge toward the lateral side or medial side. The fixation material may applied to the femoral component before, during or after the resection, and before or during the application of the femoral component onto the femur. The femoral component is affixed or secured onto the resected surface of the femur thereby securing it to the femur (e.g., applying a fixation material to the surface for receiving fixation material and pressing the femoral component onto the femur).

Methods of preparing the femoral articular surface are well known in the art, suitable methods are disclosed in “The Adult Knee,” editors Callaghan, et al., Philadelphia, Lippincott, Williams & Wilkins, 2003.

Press fit and other fixation techniques can be employed in conjunction with the methods and prostheses according to the invention.

The patellar component is installed for example by surgically exposing the patella, by preparing a surface of the patella for implantation, for example, by cutting the patella to the dimensions of a particular patellar component and securing a patellar component to the patella, wherein the patellar component comprises a patellar component of the invention, described herein.

Methods of preparing the patellar articular surface are well known in the art, suitable methods are disclosed in “The Adult Knee,” editors Callaghan, et al., Philadelphia, Lippincott, Williams & Wilkins, 2003.

Implanting the tibial tray 10 in the tibia of a subject is accomplished by first cutting the end of the upper tibia in a shape to accommodate hybrid tibial tray 10. A tibial base plate template which has an imprint of the partition is punch fit into the end of the tibia to form, in the tibia, a slot to receive the partition such as raised shoulder 22. Cement is then applied to the peripheral surface 15 of plate 11 of hybrid tibial tray 10. Hybrid tibial tray 10 is then pressed down into the upper end of the tibia so that peripheral surface 15 rests against the tibia and central surface 16 and the partition (either stepped portion 18 or raised shoulder 22) is implanted within the tibia bone tissue for fixation to the tibia. In an alternative method, a temporary partition, such as a raised shoulder, may be placed in the slot formed in the tibia. Bone cement is then placed in the area surrounding the partition to which peripheral surface 15 of the plate 11 will attach. The partition is then removed and hybrid tibial tray 10 fixed on the upper end of the tibia as aforementioned.

As seen generally in FIG. 14 implanting hybrid tibial tray 10 in the tibia of a patient is accomplished by first cutting the end of the upper tibia 50 in a shape to accommodate hybrid tibial tray 10. A tibial base plate template 52 which has an imprint 54 of the partition is punch fit into the end of the tibia to form, in the tibia, a slot 56 to receive the partition such as raised shoulder 22. Cement is then applied to the peripheral surface 15 of plate 11 of hybrid tibial tray 10. Hybrid tibial tray 10 is then pressed down into the upper end of the tibia so that peripheral surface 15 rests against the tibia and central surface 16 and the partition (either stepped portion 18 or raised shoulder 22) is implanted within the tibia bone tissue for fixation to the tibia, as shown in FIG. 15. In an alternative method shown in FIG. 16, a temporary partition 58, such as a raised shoulder, may be placed in the slot 56 formed in the tibia. Bone cement is then placed in the area surrounding the partition to which peripheral surface 15 of the plate 11 will attach. The partition 58 is then removed and hybrid tibial tray 10 fixed on the upper end of the tibia as aforementioned.

### EXAMPLE

The following example present results of laboratory tests conducted comparing the prosthesis of the invention with standard modes of prosthetic fixation such as cement, cancellous screws, central stem and cementless.

### Example 1

A uniform density polyurethane foam was used as a substrate for this study, Last-A-Foam (Pacific Plastics Research Laboratories, Vashon Island, Wash.). Its material properties are similar to tibial cancellous bone, and its use is well documented. The foam was machined into uniform blocks and each block was fitted to a testing jib to prevent variability between testing sequences and eliminate background motion artifact.

The testing jig consisted of liquid mercury strain gauges (LMSG, Parks Medical Electronics, Beaverton, Oreg.) attached to translatable arms (X, Y, Z) for alignment with the tibial tray. Each LMSG was attached to a translatable arm and attached to the tibial tray. Four LMSGs were used per tray fixed to the anterior, posterior, medial, and lateral regions of the tray. The LMSG records a voltage change due to movement of the tray. Calibration curves for each LMSG allow extrapolation of the movement in micrometers. Calibration curves were obtained by opening the gauge in fixed metric increments and recording the voltage at these increments.

The traditional noncemented trays had a 30 mm central stem. For implantation into the foam a hole was drilled, slightly smaller than the stem, to fix the prosthesis to the foam. The noncemented design with screws had two ¼″ drill holes made in the foam for fixation of the 6.5 mm cancellous screws. In addition to the central hole drilled as in the traditional noncemented. The noncemented with a long stem, had a hole drilled to accommodate the longer stem. The traditional cemented was implanted using surgical cement with 1/16″ drill holes made in the surface of the foam to mimic the operative situation and increase stability. The hybrid noncemented was fit to the block by machining an area for the undersurface to sit in the foam. The hybrid cemented also had a machined area for the undersurface. In addition, 1/16″ drill holes were made around the periphery where the cement was placed to aid in stability.

Tibial baseplates were fitted to the foam blocks with their respective modes of fixation and then centrally loaded with 150 lb. This load stabilized the tray in the foam for testing purposes. Once the tray was fixed to the foam in its respective manner it was put through the testing sequence. All specimens were loaded on a Materials Testing System (MTS Systems Corporation, Minneapolis, Minn.). Each loading trial consisted of five loads (100 lbs.-500 lbs.) in 100 pound increments, placed in one of five positions (anterior, posterior, medial, lateral, central). After each of the five loads was applied in the positions, the specimen was removed from the foam and fixed to a new block of foam. At the loading intervals, the voltage of all four LMSGs was recorded simultaneously. The load was applied, allowed to stabilize, and then sampling of the LMSGs occurred for five seconds at five hertz. This gave twenty-five data points per gauge per loading interval. These twenty-five points were averaged for each LMSG. In all trials the load was applied in a uniform axial manner, parallel to the tibia.

Each method of fixation was tested in six different blocks of foam consecutively. After all six trials were complete, the data was averaged for that method of fixation and compared to the others. Therefore, there were thirty-six blocks of foam tested in all, six configurations for six trials each. For each trial, one per foam block, a data file was created that contained the displacement of each gauge per loading situation. This allowed the comparison of all designs at all loading configurations.

Tray configurations were analyzed by an analysis of variance using a standard T-test and Tukey's studentized range (HSD) test. All configurations consisted of a Richards Genesis large right tibial component (Richards Medical Co., Memphis, Tenn.). The same noncemented component was used repeatedly depending on the configuration. The cemented component was used for the cemented applications only. Howmedica Simplex-P Radiopaque Bone Cement (Howmedica Inc. Rutherford, N.J.) was used for all trials of the cemented and the hybrid cemented. No cement centrifugation or vacuum mixing was employed.

The hybrid configuration consisted of a noncemented baseplate with a smaller 6 mm block placed on the undersurface of the tray. This provided a 5 mm rim for cement application but allowed for central ingrowth. The block was held in place through screws affixed to the plate.

In total six configurations were tested: (1) traditional cemented; (2) hybrid cemented; (3) noncemented with no additional fixation; (4) noncemented with central stem; (5) noncemented with two 6.5 mm cancellous screws; (6) hybrid noncemented.

The greatest amount of micromotion was detected at 500 pounds. The greatest micromotion (subsidence) occurred at the point of load application, except in the case of central loading which showed maximum subsidence anteriorly.

The largest subsidence values were recorded for the anterior load. At 500 pounds the values ranged from 0.407 mm to 0.724 mm. Analysis of variance demonstrated three statistically distinct groups: (1) Hybrid cemented (N=36, DOF Model=5, DOF Corrected=35, p=0.0001, F=26.85, .alpha.=0.05, r.sup.2=0.817); (2) Traditional cemented and noncemented with stem; and (3) the remaining three configurations.

Smaller values for subsidence was seen in the posterior load testing mode (range 0.192 mm to 0.366 mm). Analysis of variance demonstrated two statistically significant groups: (1) Hybrid cemented and (2) all other groups (N=36, DOF Model=5, DOF Corrected=35, p=0.0001, F=35.52, .alpha.=0.05, r.sup.2=0.855).

Subsidence values ranged from 0.213 mm to 0.413 mm for this testing model. Statistically, two separate groups were superior by analysis of variance: (1) Traditional cemented and hybrid cements, and (2) all other designs (N=36, DOF Model=5, DOF Corrected=35, p=0.0014, F=5.26, .alpha.=0.05, r.sup.2=0.467).

In this lateral load testing mode, the values ranged from 0.288 mm to 0.487 mm. Three statistically separate groups were identified by analysis of variance: (1) Traditional cemented and Hybrid cemented (N=36, DOF Model=5, DOF Corrected=35, p=0.0001, F=7.48, .alpha.=0.05, r.sup.2=0.555); (2) Hybrid noncemented (p<0.001): (3) the three remaining configurations.

Subsidence values ranged from 0.234 mm to 0.446 mm for central Loading. Analysis of variance identified two statistically different groups: (1) Traditional cemented and Hybrid cemented (N=36, DOF Model=5, DOF Corrected=35, p=0.0001, F=23.52, .alpha.=0.05, r²=0.798) and (2) the remaining four configurations.

The results of the test indicate that the hybrid tibial tray of the present invention provided equal and in some cases better initial fixation then the cemented design. In in vivo, the tibial tray of the present invention, with central ingrowth, would exhibit enhanced stability. Central bony ingrowth would account for long term stability of the prosthesis. The hybrid design permits a large undersurface area for ingrowth in addition to the use of cement for initial fixation.

### Example 2

A uniform density polyurethane foam is used as a substrate for this study, Last-A-Foam (Pacific Plastics Research Laboratories, Vashon Island, Ish.). Its material properties are similar to femoral cancellous bone, and its use is well documented. The foam is machined into uniform blocks and each block is fitted to a testing jib to prevent variability between testing sequences and eliminate background motion artifact.

The testing jig consisted of liquid mercury strain gauges (LMSG, Parks Medical Electronics, Beaverton, Oreg.) attached to translatable arms (X, Y, Z) for alignment with the femoral component. Each LMSG is attached to a translatable arm and attached to the femoral component. Four LMSGs are used per component fixed to the anterior, posterior, medial, and lateral regions of the component. The LMSG records a voltage change due to movement of the component. Calibration curves for each LMSG allow extrapolation of the movement in micrometers. Calibration curves are obtained by opening the gauge in fixed metric increments and recording the voltage at these increments.

Femoral components are fitted to the foam blocks with their respective modes of fixation and then centrally loaded with 150 lb. This load stabilized the component in the foam for testing purposes. Once the component is fixed to the foam in its respective manner it is put through the testing sequence. All specimens are loaded on a Materials Testing System (MTS Systems Corporation, Minneapolis, Minn.). Each loading trial consists of five loads (100 lbs.-500 lbs.) in 100 pound increments, placed in one of five positions (anterior, posterior, medial, lateral, central). After each of the five loads is applied in the positions, the specimen is removed from the foam and fixed to a new block of foam. At the loading intervals, the voltage of all four LMSGs are recorded simultaneously. The load is applied, allowed to stabilize, and then sampling of the LMSGs occurs for five seconds at five hertz. This gave twenty-five data points per gauge per loading interval. These twenty-five points are averaged for each LMSG. In all trials the load is applied in a uniform axial manner, parallel to the tibia.

Each method of fixation is tested in six different blocks of foam consecutively. After all six trials are complete, the data is averaged for that method of fixation and compared to the others. Therefore, there are thirty-six blocks of foam tested in all, six configurations for six trials each. For each trial, one per foam block, a data file is created that contained the displacement of each gauge per loading situation. This allowed the comparison of all designs at all loading configurations.

Component configurations are analyzed by an analysis of variance using a standard T-test and Tukey's studentized range (HSD) test. The same noncemented component is used repeatedly depending on the configuration. The cemented component is used for the cemented applications only. Howmedica Simplex-P Radiopaque Bone Cement (Howmedica Inc. Rutherford, N.J.) is used for all trials of the cemented and the hybrid cemented.

The hybrid configuration consists of a noncemented component with a smaller 6 mm block placed on the undersurface of the component. This provided a 5 mm rim for cement application but allowed for central ingrowth. The block is held in place through screws affixed to the plate.

In total six configurations are tested: (1) traditional cemented; (2) hybrid cemented; (3) noncemented with no additional fixation; (4) noncemented with stems; (5) noncemented with cancellous screws; (6) hybrid noncemented.

The results of the test will indicate that the hybrid femoral component of the present invention provides equal and in some cases better initial fixation than the cemented design. In vivo, the femoral component of the present invention, with central ingrowth, would exhibit enhanced stability. Central bony ingrowth would account for long term stability of the prosthesis. The hybrid design permits a large undersurface area for ingrowth in addition to the use of cement for initial fixation.

While the invention has been disclosed with reference to specific embodiments, it is apparent that other embodiments and variations of the invention may be devised by others skilled in the art without departing from the true spirit and scope of the invention. The appended claims are intended to be construed to include all such embodiments and equivalent variations.

What is claimed is:

1. A knee replacement prosthesis comprising:
a femoral component of generally concave shape comprising:
a bone facing surface and an anatomically shaped articular surface having anterior and posterior ends, wherein the posterior end forms two condyles along an axis of the femoral component, wherein the bone facing surface comprises at least one stem, a surface for receiving a fixation material adjacent to the anterior and posterior ends, and a central surface for promoting bone ingrowth.

2. The knee replacement prosthesis of claim 1, further comprising:
a tibial component comprising:
a laterally extending plate having a proximal surface and a distal surface, the distal surface including a peripheral surface for receiving fixation material and a planar central surface which promotes bone ingrowth, the peripheral surface and the planar central surface each having surface areas which are substantially equal; and
a central stem extending from the distal surface and being substantially perpendicular thereto.

3. The knee replacement prosthesis of claim 1, further comprising:
a patellar component comprising:
a substantially circular plate having a patellar facing surface, wherein the patellar facing surface has a peripheral surface for receiving a fixation material and a central surface for promoting bone ingrowth.

4. The knee replacement prosthesis of claim 1, further comprising:
a patellar component comprising:
a substantially circular plate having a patellar facing surface, wherein the patellar facing surface has a peripheral surface for promoting bone ingrowth and a central surface for receiving a fixation material.

5. The knee replacement prosthesis of claim 1, further comprising:
a patellar component comprising:
a substantially circular plate having a patellar facing surface, wherein the patellar facing has a one or more pegs and a surface for promoting bone ingrowth.

6. The knee replacement prosthesis of claim 1, wherein the planar central surface and the central surface have bone ingrowth promoting material.

7. The knee replacement prosthesis of claim 6, wherein the material is a porous layer.

8. The knee replacement prosthesis of claim 7, wherein the porous layer is composed of a plurality of beads.

9. The knee replacement prosthesis of claim 7, wherein the porous layer has a thickness in a range of 0.5 to 2.0 mm.

10. The knee replacement prosthesis of claim 1, wherein the surface for receiving a fixation material and the central surface of the femoral component are substantially equal in area.

11. The knee replacement prosthesis of claim 1, wherein the surface for receiving a fixation material is about ⅓ to about ½ of the one facing surface.

12. The knee replacement prosthesis of claim 1, wherein surface for receiving a fixation material is a recessed pocket.

13. The knee replacement prosthesis of claim 1, wherein the recessed pocket provides additional surface area and improved adhesion when the prosthesis is implanted against a bone surface.

14. The knee replacement prosthesis of claim 1, wherein the femoral component further comprises an anterior partition and a posterior partition on each condyle, wherein each partition separates the surfaces for receiving a fixation material from the central surface.

15. The knee replacement prosthesis of claim 2, wherein the tibial component further comprises a partition continuously extending around the planar central surface which separates the peripheral surface from the planar central surface.

16. The knee replacement prosthesis of claim 15, wherein the partition has a V-shaped cross section.

17. The knee replacement prosthesis of claim 15, wherein the partition is a raised shoulder having a height in a range of 6.0 to 14.0 mm.

18. The knee replacement prosthesis of claim 15, wherein the partition is a stepped portion raised in relation to the peripheral surface and containing the planar central surface.

19. The knee replacement prosthesis of claim 18, wherein the stepped portion has a height in a range of 3.0 to 8.0 mm.

20. The knee replacement prosthesis of claim 18, wherein the stepped portion has a height of 6.0 mm.

21. The knee replacement prosthesis of claim 2, wherein the laterally extending plate has a height in a range of 3.0 to 7.0 mm.

22. The knee replacement prosthesis of claim 2, wherein the laterally extending plate has a width of 80.0 mm and a length of 53.0 mm.

23. A knee replacement prosthesis comprising:
a femoral component of generally concave shape comprising:
a bone facing surface and an anatomically shaped articular surface having anterior and posterior ends, wherein the posterior end forms two condyles along an axis of the femoral component, wherein the bone facing surface comprises at least one stem, a surface for receiving a fixation material along a perimeter of the bone facing surface and a central surface for promoting bone ingrowth.

24. The knee replacement prosthesis of claim 23, comprising:
a tibial component comprising:
a laterally extending plate having a proximal surface and a distal surface, the distal surface including a peripheral surface for receiving fixation material and a planar central surface which promotes bone ingrowth, the peripheral surface and the planar central surface each having surface areas which are substantially equal; and
a central stem extending from the distal surface and being substantially perpendicular thereto.

25. The knee replacement prosthesis of claim 23, further comprising:
a patellar component comprising:
a substantially circular plate having a patellar facing surface, wherein the patellar facing surface has a peripheral surface for receiving a fixation material and a central surface for promoting bone ingrowth.

26. The knee replacement prosthesis of claim 23, further comprising:
a patellar component comprising:
a substantially circular plate having a patellar facing surface, wherein the patellar facing surface has a peripheral surface for promoting bone ingrowth and a central surface for receiving a fixation material.

27. The knee replacement prosthesis of claim 23, further comprising:
a patellar component comprising:
a substantially circular plate having a patellar facing surface, wherein the patellar facing has a one or more pegs and a surface for promoting bone ingrowth.

28. The knee replacement prosthesis of claim 23, wherein the femoral component further comprises a partition continuously extending around a perimeter of the bone facing surface which separates the surface for receiving a fixation material and the central surface.

29. The knee replacement prosthesis of claim 28, wherein the partition is adapted to prevent fixation material from flowing into the central surface.

30. The knee replacement prosthesis of claim 28, wherein the partition has a V-shaped cross section.

31. The knee replacement prosthesis of claim 28, wherein the partition is a raised shoulder having a height in a range of 6.0 to 14.0 mm.

32. The knee replacement prosthesis of claim 28, wherein the partition is a stepped portion raised in relation to the peripheral surface and containing the planar central surface.

33. The knee replacement prosthesis of claim 32, wherein the stepped portion has a height in a range of 3.0 to 8.0 mm.

34. The knee replacement prosthesis of claim 32, wherein the stepped portion has a height of 6.0 mm.

35. The knee replacement prosthesis of claim 23, wherein the planar central surface and the central surface have bone ingrowth promoting material.

36. The knee replacement prosthesis of claim 35, wherein the bone ingrowth promoting material is a porous layer.

37. The knee replacement prosthesis of claim 36, wherein the porous layer is composed of a plurality of beads.

38. The knee replacement prosthesis of claim 36, wherein the porous layer has a thickness in a range of 0.5 to 2.0 mm.

39. The knee replacement prosthesis of claim 23, wherein the surface for receiving a fixation material and the central surface of the femoral component are substantially equal in area.

40. The knee replacement prosthesis of claim 23, wherein the surface for receiving a fixation material is about ⅓ to about ½ of the bone facing surface.

41. The knee replacement prosthesis of claim 23, wherein surface for receiving a fixation material is a recessed pocket.

42. A knee replacement prosthesis comprising:
a patellar component comprising:
a substantially circular plate having a patellar facing surface, wherein the patellar facing surface has a peripheral surface for receiving a fixation material and a central surface for promoting bone ingrowth.

43. A knee replacement prosthesis comprising:
a patellar component comprising:
a substantially circular plate having a patellar facing surface, wherein the patellar facing surface has a peripheral surface for promoting bone ingrowth and a central surface for receiving a fixation material.

44. The knee replacement prosthesis of claim 3, further comprising a partition continuously extending around the circular plate separating the peripheral surface from the planar central surface.

45. The knee replacement prosthesis of claim 44, wherein the partition has a V-shaped cross section.

46. The knee replacement prosthesis of claim 44, wherein the partition is a raised shoulder having a height in a range of 6.0 to 14.0 mm.

47. The knee replacement prosthesis of claim 44, wherein the partition is a stepped portion raised in relation to the peripheral surface and the central surface.

48. The knee replacement prosthesis of claim 47, wherein the stepped portion has a height in a range of 3.0 to 8.0 mm.

49. The knee replacement prosthesis of claim 47, wherein the stepped portion has a height of 6.0 mm.

50. The knee replacement prosthesis of claim 44, wherein the partition is adapted to prevent fixation material from flowing between the central surface and peripheral surface.

51. The knee replacement prosthesis of claim 3, wherein the surface for receiving a fixation material and the central surface are substantially equal in area.

52. The knee replacement prosthesis of claim 3, wherein the surface for receiving a fixation material is about ⅓ to about ½ of the bone facing surface.

53. The knee replacement prosthesis of claim 3, wherein surface for receiving a fixation material is a recessed pocket.

54. A knee replacement prosthesis comprising:
a patellar component comprising:
a substantially circular plate having a patellar facing surface, wherein the patellar facing has a one or more pegs and a surface for promoting bone ingrowth.

55. The knee replacement prosthesis of claim 54, wherein the pegs are attached to the bone with a fixation material.

56. The knee replacement prosthesis of claim 3, wherein the bone ingrowth promoting material is a porous layer.

57. The knee replacement prosthesis of claim 56, wherein the porous layer is composed of a plurality of beads.

58. The knee replacement prosthesis of claim 57, wherein the porous layer has a thickness in a range of 0.5 to 2.0 mm.

59. The knee replacement prosthesis of claim 3, wherein the circular plate has a height in a range of 3.0 to 7.0 mm.

60. The knee replacement prosthesis of claim 3, wherein the circular plate has a circumference of between about 12 to about 40 mm.

61. A method for implanting knee replacement prosthesis, comprising:
surgically exposing the knee joint of a subject,
preparing the surface of the femur for implantation, and
securing a femoral component of generally concave shape to the femur of the subject, wherein the femoral component comprises a bone facing surface and an anatomically shaped articular surface having anterior and posterior ends, wherein the posterior end forms two condyles along an axis of the femoral component, wherein the bone facing surface comprises at least one stem, a surface for receiving a fixation material adjacent to the anterior and posterior ends, and a central surface for promoting bone ingrowth.
